(19) United States
(12) Patent Application Publication
(10) Pub. No.: US 2013/0296965 A1
(43) Pub. Date: Nov. 7, 2013

(54) METHOD FOR BLOOD PRESSURE MODULATION USING ELECTRICAL STIMULATION OF THE CORONARY BARORECEPTORS

(71) Applicant: CARDIAC PACEMAKERS, INC., St. Paul, MN (US)

(72) Inventors: Eric A. Mokelke, White Bear Lake, MN (US); Allan C. Shuros, St. Paul, MN (US); Jason J. Hamann, Blaine, MN (US); William Conrad Stoffregen, Lake Elmo, MN (US)

(73) Assignee: Cardiac Pacemakers, Inc., St. Paul, MN (US)

(21) Appl. No.: 13/832,486

(22) Filed: Mar. 15, 2013

Related U.S. Application Data
(60) Provisional application No. 61/643,698, filed on May 7, 2012.

(51) Int. Cl. A61N 1/36

(52) U.S. Cl. CPC A61N 1/36135
USPC 607/44

(57) ABSTRACT

An apparatus comprises a first stimulation circuit and a control circuit. The stimulation circuit is configured to be electrically coupled to a first electrode assembly that is configured to deliver electrical sub-myocardial activation stimulation to a coronary baroreceptor from a location within a left atrial appendage of a heart. The stimulation circuit is further configured to generate the electrical stimulation for delivery to the coronary baroreceptor via the first electrode assembly. The control circuit is wirelessly or conductively coupled to the first stimulation circuit and is configured to control delivery of the electrical stimulation.

### CLAIM OF PRIORITY

This application claims the benefit of priority under 35 U.S.C. §119(e) of Mokelke et al., U.S. Provisional Patent Application Ser. No. 61/643,698, entitled “METHOD FOR BLOOD PRESSURE MODULATION USING ELECTRICAL STIMULATION OF THE CORONARY BARORECEPTORS”, filed on May 7, 2012, which is herein incorporated by reference in its entirety.

### BACKGROUND

Electrical stimulation therapy has been proposed as a therapy for a number of conditions. Examples of stimulation therapies include electrical stimulation therapies for respiratory problems such as sleep disordered breathing, cardiac rhythm management, myocardial infarction and ischemia, heart failure (HF), epilepsy, depression, pain, migraines, eating disorders and obesity, movement disorders, and blood pressure control such as to treat hypertension.

Hypertension is a cause of heart disease and other related cardiac co-morbidities. Hypertension occurs when blood vessels constrict. As a result, the heart works harder to maintain flow at a higher blood pressure, which can contribute to HF. Hypertension generally relates to high blood pressure, such as a transitory or sustained elevation of systemic arterial blood pressure to a level that is likely to induce cardiovascular damage or other adverse consequences. Hypertension has been defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, myocardial infarction, dissecting aneurysm, and renovascular disease. A large segment of the general population, as well as a large segment of patients implanted with pacemakers or defibrillators suffer from hypertension. The long term mortality as well as the quality of life can be improved for this population if their blood pressure and hypertension can be reduced. Many patients who suffer from hypertension do not respond to treatment, such as treatments related to lifestyle changes and hypertension drugs.

### OVERVIEW

This document relates generally to systems, devices, and methods that provide electrical stimulation therapy to a patient or subject. In particular it relates to systems, devices, and methods to treat hypertension using electrical stimulation therapy.

An apparatus example includes a first stimulation circuit and a control circuit. The stimulation circuit is configured to be electrically coupled to a first electrode assembly that is configured to deliver electrical sub-myocardial activation stimulation to a coronary baroreceptor or coronary baroreceptor rich area from a location within a left atrial appendage of a heart. The stimulation circuit is further configured to generate the electrical stimulation for delivery to the coronary baroreceptor via the first electrode assembly. The control circuit is wirelessly or conductively coupled to the first stimulation circuit and is configured to control delivery of the electrical stimulation.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 is a block diagram of portions of a medical device that delivers electrical stimulation therapy to one or more coronary baroreceptors of a heart to reduce hypertension.

FIG. 2 is an illustration of an example of a medical device that can be used to treat hypertension.

FIG. 3 is an illustration of portions of an example of a system that can be used to treat hypertension by applying electrical stimulation to coronary baroreceptors.

FIG. 4 is an illustration of portions of another example of a system that can be used to treat hypertension.

FIG. 5 is a flow diagram of a process to produce a stimulation device that can be used to treat hypertension.

### DETAILED DESCRIPTION

This document discusses systems and methods for delivering electrical stimulation therapy. A medical device can include one or more of the features, structures, methods, or combinations thereof described herein. For example, an electrical neurostimulator may be implemented to include one or more of the advantageous features or processes described below. Such a device may be implemented to provide a variety of therapeutic or diagnostic functions.

The autonomic nervous system (ANS) regulates “involuntary” organs, while the contraction of voluntary (skeletal) muscles is controlled by somatic motor nerves. Examples of involuntary organs include respiratory and digestive organs, and also include blood vessels and the heart. Often, the ANS functions in an involuntary, reflexive manner to regulate glands, to regulate muscles in the skin, eye, stomach, intestines and bladder, and to regulate cardiac muscle and the muscles around blood vessels, for example.

The ANS includes the sympathetic nervous system and the parasympathetic nervous system. The sympathetic nervous system is affiliated with stress and the “fight or flight response” to emergencies. Among other effects, the “fight or flight response” increases blood pressure and heart rate to increase skeletal muscle blood flow, and decreases digestion to provide the energy for “fighting or fleeing.” The parasympathetic nervous system is affiliated with relaxation and the “rest and digest response” which, among other effects, decreases blood pressure and heart rate, and increases digestion to conserve energy. The ANS maintains normal internal function and works with the somatic nervous system. Afferent nerves convey impulses toward a nerve center, and efferent nerves convey impulses away from a nerve center.

Stimulating the sympathetic and parasympathetic nervous systems can cause changes in heart rate, blood pressure and other physiological responses. Neurostimulation to treat cardiovascular diseases can be referred to as neurocardiac therapy (NCT). NCT, by way of example and not limitation, includes the stimulation of an autonomic neural target to provide a therapy for a cardiac arrhythmia, ischemia, heart failure, angina, atherosclerosis, blood pressure, and the like. By way of example and not limitation, autonomic neural targets used to deliver NCT include the vagus nerve, cardiac branches of the vagal nerves, the carotid sinus nerve, chemoreceptors, cardiac fat pads, the spinal column, some nerve roots extending from the spinal column, or baroreceptors such as coronary baroreceptors, baroreceptors in the carotid sinus, or baroreceptors in the pulmonary artery.

Baroreflex refers to a reflex triggered by stimulation of a baroreceptor. Baroreceptors include stretch receptors that are sensitive to stretching of an arterial wall resulting from increased pressure from within. Stimulation of baroreceptors inhibits sympathetic nerve activity and reduces systemic perfusion pressure by decreasing peripheral vascular resistance, cardiac contractility, and reduces or potentially reduces heart rate.

Coronary baroreceptors are naturally stimulated by internal pressure and the stretching of the wall of a coronary vessel. The coronary baroreceptors may be artificially stimulated by electrical stimulation, which can lead to reduction in systemic blood pressure. Some coronary baroreceptors are located within a small area around the coronary artery and located very proximally in the left anterior descending (LAD) artery and the circumflex (CFX) artery. The sensitivity of the coronary baroreceptors to stimulation may be higher than that of the carotid sinus baroreceptors. Hence, stimulation of the coronary baroreceptors may provide better control of blood pressure.

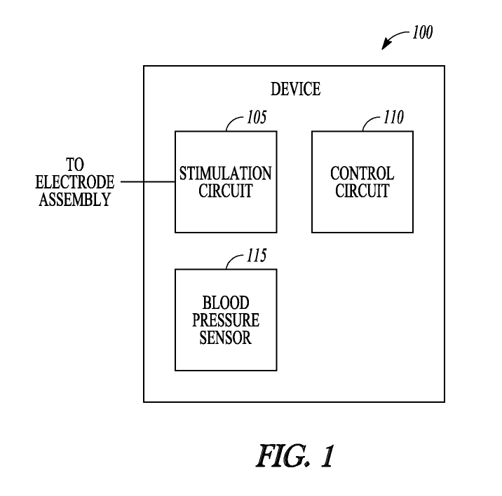

FIG. 1 is a block diagram of portions of a medical device that delivers electrical stimulation therapy to one or more coronary baroreceptors of a heart to reduce hypertension. The device 100 includes a stimulation circuit 105 and a control circuit 110. The stimulation circuit 105 is configured to be electrically coupled to an electrode assembly (not shown). The stimulation circuit 105 generates the electrical stimulation energy that is delivered via the electrode assembly.

The control circuit 110 can be a processor, a digital signal processor (DSP), application specific integrated circuit (ASIC), microprocessor, or other type of processor, interpreting or executing instructions in software modules or firmware modules. The control circuit 110 can be a sequencer circuit. A sequencer refers to a state machine or other circuit that sequentially steps through a fixed series of steps to perform one or more functions. The steps are typically implemented in hardware or firmware. The control circuit 110 can include other circuits or sub-circuits to perform the functions described. These circuits may include software, hardware, firmware or any combination thereof. Multiple functions can be performed in one or more of the circuits as desired. In some examples, the control circuit 110 is conductively coupled to the neurostimulation circuit 105. In some examples, the control circuit is wirelessly coupled to the neurostimulation circuit 105.

The control circuit 110 controls the delivery of the stimulation to the coronary baroreceptors. In some examples, the control circuit 110 adjusts the stimulation at least in part in response to sensed information indicative of blood pressure. This can result in a closed loop system where the control circuit 110 adjusts delivery of electrical stimulation to achieve a target blood pressure. For instance, the control circuit 110 may increase stimulation of the coronary baroreceptors to decrease systemic perfusion pressure and may decrease stimulation of the baroreceptors to increase systemic blood perfusion in order to modulate blood pressure to a specified value or to maintain the pressure within a target range of pressure values. In some examples, the device 100 includes an implantable blood pressure sensor 115 that provides information indicative of blood pressure to the control circuit 110. In some examples, the blood pressure sensor is configured (e.g., shaped and sized) for implantation in a coronary vessel to provide a direct measure of vessel pressure. In some examples, the blood pressure sensor is configured for implantation in the pulmonary artery. In some examples, the blood pressure is located external to the patient and communicates blood pressure information wirelessly to the control circuit 110. In some examples, the control circuit 110 receives blood pressure information from another type of separate implantable device.

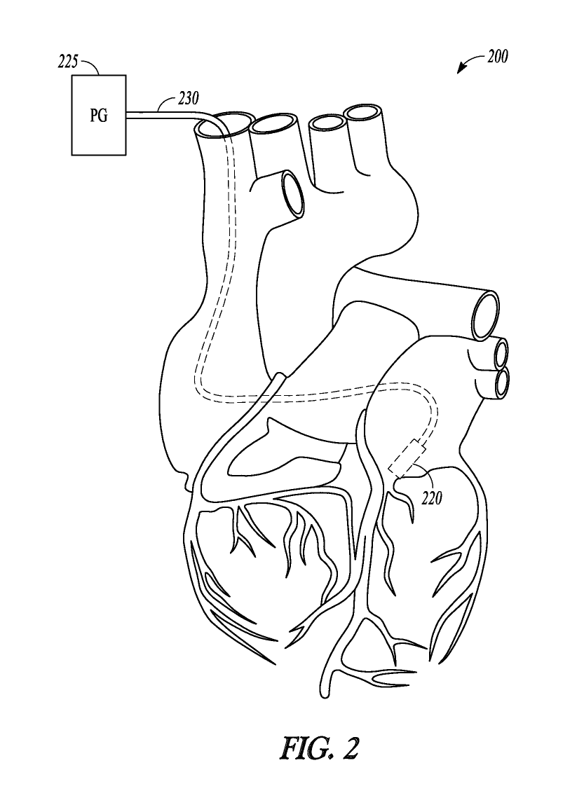

FIG. 2 is an illustration of an example of a medical device that can be used to treat hypertension by applying electrical stimulation from a location within a left atrial appendage of a heart of a subject. The left atrial appendage is a muscular pouch connected to the left atrium and functions as a reservoir for the left atrium. The device 200 includes an electrode assembly 220 and a pulse generator 225 that includes an electronics unit such as the device described in FIG. 1. In some examples, the pulse generator 225 includes a subcutaneously-implantable housing and the control circuit and neurostimulation circuit are included within the subcutaneously-implantable housing.

The electrode assembly 220 is configured to deliver electrical sub-myocardial activation stimulation to a coronary baroreceptor. This sub-threshold stimulation prevents inducement of cardiac depolarization from the left atrium. In some examples, the stimulation circuit generates electrical stimulation pulses that have low enough amplitude or a narrow enough pulse width so that the stimulation pulses do not induce cardiac depolarization. In certain examples, the pulse width is about 0.3 milliseconds (msec) and the amplitude of the stimulation energy is in the range of about five to six volts. The stimulation circuit is configured to provide an electrical stimulation therapy specified to assist a blood pressure reduction at least in part via an inhibition of the sympathetic nervous system. The inhibition of the sympathetic nervous system is induced via the sub-myocardial activation stimulation of one or more coronary baroreceptors or a baroreceptor rich area.

The coronary baroreceptors can be stimulated by the device 100 at anytime during a cardiac cycle and the stimulation does not have to limited to certain points of the cardiac cycle (e.g., limited to a refractory period). Chronaxie refers to the minimum time that an electrical pulse needs to be applied to cause stimulation. Chronaxie is lower for nervous tissue than for myocardial tissue, so an electrical pulse that fails to stimulate myocardial tissue can still be effective to stimulate nervous tissue. Because chronaxie is lower in nervous tissue versus myocardial tissue, neurostimulation of coronary baroreceptors can be provided during non-refractory periods. This can simplify the design of the device 200 because the cardiac cycle does not have to be sensed to coordinate delivery of electrical stimulation therapy with the cardiac cycle.

According to some examples, the stimulation of the coronary baroreceptors is timed to specific events in the cardiac cycle, such as diastole for example. During diastole, blood flow increases in microvessels and therefore there may be more stretching of baroreceptors. In some examples, the stimulation of the coronary baroreceptors is timed to occur during systole, when there is significant blood flow in conduit vessels.

In some examples, the electrode assembly 220 can be electrically coupled to the stimulation circuit, such as by an intravascularly-deliverable lead assembly 230. In some examples, the distal portion of the lead assembly 230 can be configured by size and shape for transseptal delivery to the left atrial appendage of the heart via the septal region between the right atrium and the left atrium of the heart as shown in the FIG. 2. The electrode assembly 220 can include a fixation device to securely anchor at least a portion of the electrode assembly within the left atrial appendage. Some examples, of such fixation devices include, among other things, one or more hooks, tines, or a screw tip incorporated into the electrode assembly 220, or the electrode assembly 220 can be retained in the left atrial appendage by way of a spring mechanism or balloon. In some examples, the electrode assembly 220 includes a pressure sensor. In some examples, the device includes an implantable pressure sensor separate from the electrode assembly, such as in the lead assembly 230 attached to the electrode or in a separate lead assembly conductively coupled to the pulse generator 225.

Other deployments of stimulation devices can be useful as well. In some examples, the distal portion of the lead assembly 230 is sized and shaped to be deployed in a coronary vein and positioned adjacent to the location of coronary baroreceptors. In certain examples, the distal portion of the lead assembly is sized and shaped for deployment in a coronary artery. In certain examples, the lead assembly is configured for epicardial placement. For some applications, the device 200 can include multiple leads. For instance, the device 200 can include a first electrode and lead assembly sized and shaped for transseptal delivery to the left atrial appendage of the heart and a second lead assembly sized and shaped to be deployed in a coronary vein. For such situations, the control circuit may be configured (e.g., programmed) to selectively provide electrical stimulation using one or both of the electrode-lead assemblies.

The device 200 may be a standalone system or can be integrated with one or more other stimulation systems, such as a system to provide electrical stimulation to baroreceptors in the area of the carotid sinus or aortic arch. In certain examples, the systems communicate wirelessly (e.g., using radio frequency or inductive telemetry) to coordinate delivery of the stimulation to one or more baroreceptor locations, which can include a non-coronary baroreceptor location. The delivery of electrical stimulation to the coronary and non-coronary baroreceptors may be synergistic and provide blood pressure reduction using multiple sets of baroreceptors, or the stimulation delivery can be isolated to either the coronary or non-coronary location. In certain examples, the device 200 is included in a cardiac rhythm management (CRM) system that provides electrical stimulation therapy to the heart for treatment of cardiac arrhythmia.

In some cases, the device 200 may be leadless. For instance, the electrode assembly 220 and the stimulation circuit may be included as a portion of a wirelessly-controlled electrode assembly configured by shape and size to be located entirely within the heart (e.g., within the left atrial appendage). The control circuit can be located remotely from the wirelessly-controlled electrode assembly and can be wirelessly coupled to the stimulation circuit to control the stimulation. The wirelessly-controlled electrode assembly may be deployed via catheter and may include a fixation device to anchor at least a portion of the wirelessly-controlled electrode assembly to the left atrial appendage. The fixation device may include a screw tip or an occlusion device such as a mesh or web. The control circuit may be included in a subcutaneously-implantable housing separate from the wirelessly-controlled electrode assembly or can be included in an external wearable device. The wirelessly-controlled electrode assembly may include a pressure sensor and the wirelessly-controlled electrode assembly communicates sensed information indicative of blood pressure to the control circuit.

In certain examples, the leadless device may include a second wirelessly-controlled electrode assembly that includes a second stimulation circuit. The second wirelessly-controlled electrode assembly may be deployed to deliver electrical stimulation to another coronary baroreceptor location or a non-coronary baroreceptor location. The control circuit may wirelessly control generation of electrical stimulation by the second electrode assembly in coordination with initiating or inhibiting generation of electrical stimulation using the first wirelessly-controlled electrode assembly. In some examples, the leadless device may be included in an intracavity pacemaker for implantation in the left atrial appendage.

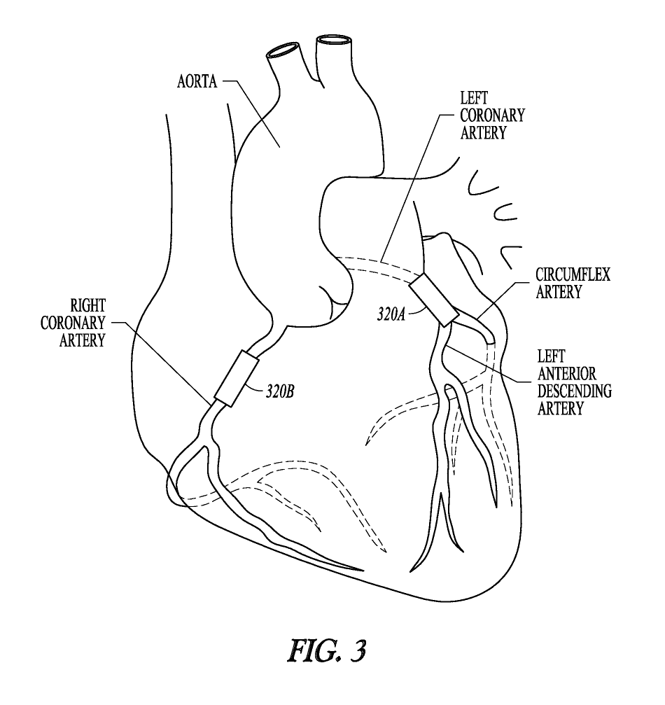

FIG. 3 is an illustration of portions of an example of a system that can be used to treat hypertension by applying electrical stimulation to coronary baroreceptors. In the example, the electrode assembly and the stimulation circuit are included in one or more stent-type stimulators 320A, 320B. A stent-type stimulator can be deployed in one or both of the left coronary artery or the right coronary artery near a location of coronary baroreceptors. In some examples, a stent-type stimulator can be deployed in the left atrial appendage. A stent-type stimulator can be controlled wirelessly using a separate device that includes a controller circuit, or a controller circuit can be included in the stent-type stimulator. In some examples, the stent-type stimulator is powered by a battery or a rechargeable battery. In some examples, the stent-type stimulator is powered by wireless signals transmitted from an external device. In some examples, the stent-type stimulator includes a pressure sensor configured to sense pressure of the coronary artery in which it is implanted. The stent-type stimulator may includes a communication circuit to communicate (e.g., via radio frequency) sensed pressure information indicative of blood pressure to a separate device that includes the control circuit.

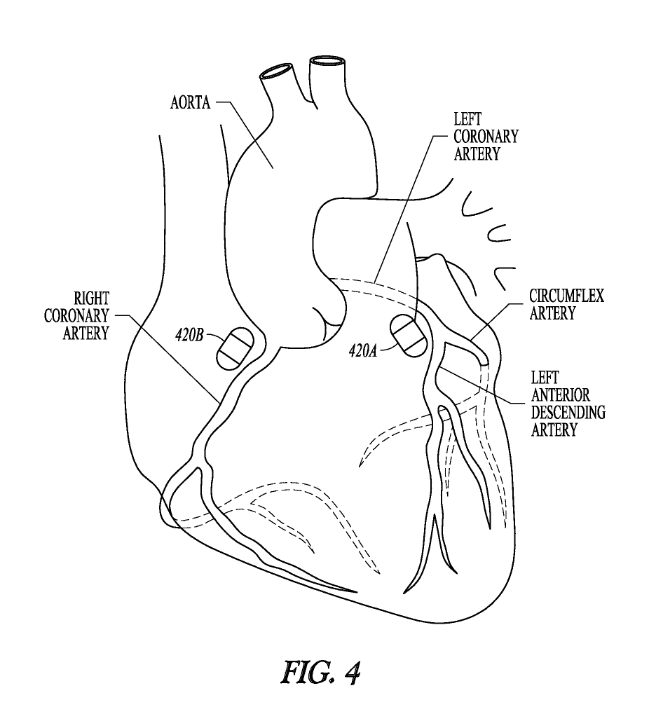

FIG. 4 is an illustration of portions of another example of a system that can be used to treat hypertension by applying electrical stimulation to coronary artery baroreceptors. The system includes one or more leadless microelectronic devices 420A, 420B deployed epicardially or intra-myocardially at or near the location of coronary baroreceptors (e.g., near baroreceptors located near the left coronary artery and the right coronary artery). An example of a leadless microelectronics device is an implantable bion microstimulator. A bion microstimulator is a miniature implantable device that can be self-contained by including a power source (e.g., a battery or rechargeable battery).

The leadless microelectronic devices 420A, 420B include a stimulation circuit and an electrode assembly. The electrode assembly can include a fixation device to anchor the microelectronic device to the myocardium. In some examples, the housing of the microelectronic device is shaped to accommodate suturing to the myocardium. The electrode assembly can include one or more electrodes formed on a subcutaneously-implantable housing of the devices. The leadless microelectronic devices 420A, 420B may include a control circuit or the leadless microelectronic devices may be wirelessly controlled by a control circuit located in a separate device.

In some examples, the leadless microelectronic devices 420A, 420B include a communication circuit to communicate with a separate device using radio frequency signals. The separate device may also be leadless microelectronic device that includes the control circuit and functions as a master device to wirelessly control other implanted devices. In some examples, the separate device may be an external controller that includes the control circuit to wirelessly control the microelectronic devices, or the separate device can be an external programmer for the microelectronic devices and the communication by radio frequency signals can be used to program or recharge the devices. In some examples, the leadless microelectronic devices 420A, 420B include pressure sensors and communicate information indicative of blood pressure to a separate device.

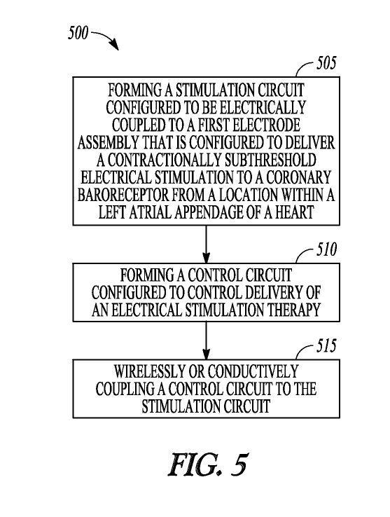

FIG. 5 is a flow diagram of a process to produce a stimulation device that can be used to treat hypertension by applying electrical stimulation to coronary artery baroreceptors. At block 505, a stimulation circuit is formed. The stimulation circuit is configured to be electrically coupled to a first electrode assembly that delivers sub-myocardial activation stimulation to a coronary baroreceptor from a location within a left atrial appendage of a heart. The stimulation circuit can be configured to generate the electrical stimulation for delivery to the coronary baroreceptor via the first electrode assembly. The stimulation circuit can provide pulsatile electrical stimulation that can be sub-myocardial activation by having one or both of a magnitude low enough and a pulse width narrow enough to avoid inducement of a cardiac contraction. In some examples, the electrical stimulation can be a train of pulses each of which is sub-myocardial activation. This allows an energy target to be delivered to the baroreceptor location while avoiding capture of the heart.

At block 510, forming a control circuit that is configured to control delivery of an electrical stimulation therapy is formed. At block 515, the control circuit is wirelessly or conductively coupled to the stimulation circuit. The control circuit can be wirelessly coupled to a stimulation circuit that resides in a stent or implantable bion device. The control circuit and the stimulation circuit can reside in the same device and the control circuit can be conductively coupled to the stimulation circuit via a conductor (e.g., wire, conductive trace) of an electrical assembly.

The devices, systems, and methods described herein provide neurostimulation to an area or areas of coronary artery baroreceptors. This neurostimulation can be used to inhibit the sympathetic nervous system to reduce blood pressure in patients with hypertension. The devices and systems described can be stand alone or work in tandem with other devices that operate to reduce blood pressure, or can be used in combination to drug therapy to reduce blood pressure in the patient. Providing electrical stimulation therapy to the coronary artery baroreceptors in combination with stimulation to sites of other baroreceptors sites can enhance the overall performance of the stimulation therapy.

### ADDITIONAL NOTES AND EXAMPLES

Example 1 can include subject matter (such as an apparatus) comprising a first stimulation circuit configured to be electrically coupled to a first electrode assembly configured to deliver an electrical sub-myocardial activation stimulation to a coronary baroreceptor from a location within a left atrial appendage of a heart, and a control circuit wirelessly or conductively coupled to the first stimulation circuit. The first stimulation circuit is configured to generate the electrical stimulation for delivery to the coronary baroreceptor via the first electrode assembly, and the control circuit is configured to control delivery of the electrical stimulation.

In Example 2, the subject matter of Example 1 can optionally include a control circuit configured to adjust the electrical stimulation at least in part in response to sensed information indicative of a blood pressure.

In Example 3, the subject matter of one or any combination of Examples 1 and 2 can optionally include an implantable blood pressure sensor configured to provide to the control circuit information indicative of blood pressure.

In Example 4, the subject matter of one or any combination of Examples 1-3 can optionally include a control circuit and a first stimulation circuit included in a subcutaneously-implantable housing. The control circuit can optionally be conductively coupled to the first stimulation circuit.

In Example 5, the subject matter of one or any combination of Examples 1-4 can optionally include an electrode assembly that includes an intravascularly-deliverable lead assembly electrically coupled to the first stimulation circuit.

In Example 6, the subject matter of Example 5 can optionally include a distal portion of the intravascularly-deliverable lead assembly sized and shaped for transseptal delivery to the left atrial appendage of the heart via a septal region between a right atrium and a left atrium of the heart.

In Example 7, the subject matter of one or any combination of Examples 1-6 can optionally include the first stimulation circuit and an electrode assembly included as a portion of a wirelessly-controlled electrode assembly configured to be located entirely within the heart, and the control circuit can optionally be located remotely from the wirelessly-controlled electrode assembly and wirelessly coupled to the first stimulation circuit.

In Example 8, the subject matter of one or any combination of Examples 1-7 can optionally include an electrode assembly that includes a fixation device configured to securely anchor at least a portion of the electrode assembly within the left atrial appendage of the heart, and the fixation device is optionally configured to locate the first electrode assembly in a specified position to deliver the electrical stimulation to the coronary baroreceptor from within the left atrial appendage of the heart.

In Example 9, the subject matter of one or any combination of Examples 1-8 can optionally include a second electrode assembly configured to deliver electrical stimulation to a non-coronary baroreceptor.

In Example 10, the subject matter of Example 9 can optionally include a second electrode assembly conductively coupled to a second stimulation circuit, and include a control circuit configured to control generation of an electrical stimulation by the second stimulation circuit for delivery via the second electrode assembly in coordination with initiation or inhibiting generation of the electrical stimulation by the first stimulation circuit.

In Example 11, the subject matter of one or any combination of Examples 1-10 can optionally include a stimulation circuit optionally configured to provide a stimulation therapy specified to assist a blood pressure reduction at least in part via an inhibition of the sympathetic nervous system induced via the electrical sub-myocardial activation stimulation.

Example 12 can include subject matter (such as an apparatus) or can optionally be combined with the subject matter of one or any combination of Examples 1-11 to include subject matter, comprising a stimulation circuit configured to be electrically coupled to an electrode assembly configured to deliver an electrical sub-myocardial activation stimulation to a coronary baroreceptor from a location within a left atrial appendage of a heart, and a control circuit wirelessly or conductively coupled to the stimulation circuit and configured to control delivery of the electrical stimulation. The stimulation circuit can optionally be configured to generate the electrical stimulation for delivery to the coronary baroreceptor via the first electrode assembly, and the stimulation circuit can optionally be configured to provide electrical stimulation therapy specified to assist a blood pressure reduction at least in part via an inhibition of the sympathetic nervous system induced via the electrical sub-myocardial activation stimulation.

Example 13 can include subject matter (such as a method, a means for performing acts, or a machine readable medium including instructions that, when performed by the machine, that can cause the machine to perform acts), or can optionally be combined with the subject matter of one or any combination of Examples 1-12 to include subject matter, comprising forming a first stimulation circuit configured to be electrically coupled to a first electrode assembly configured to deliver an electrical sub-myocardial activation stimulation to a coronary baroreceptor from a location within a left atrial appendage of a heart, the first stimulation circuit configured to generate the electrical stimulation for delivery to the coronary baroreceptor via the first electrode assembly, forming a control circuit configured to control delivery of the electrical sub-myocardial activation stimulation, and wirelessly or conductively coupling a control circuit to the first stimulation circuit. Such subject matter can include means for wirelessly coupling a control circuit to the first stimulation circuit, illustrative examples of which include coupling via radio frequency (RF) telemetry or inductive telemetry. Such subject matter can include means for conductively coupling a control circuit to the first stimulation circuit, illustrative examples of which include a conductive trace on a printed circuit board or a conductive lead.

In Example 14, the subject matter of Example 13 can optionally include receiving information indicative of a blood pressure, and adjusting an electrical stimulation therapy, using the control circuit, at least in part in response to the received information indicative of a blood pressure.

In Example 15, the subject matter of one or any combination of Examples 13 and 14 can optionally include the control circuit being conductively coupled to the first stimulation circuit, and the control circuit and the first stimulation circuit being included in a subcutaneously-implantable housing. The first electrode assembly can optionally include an intravascularly-deliverable lead assembly and can optionally be electrically coupled to the first stimulation circuit. The intravascularly-deliverable lead assembly can optionally be configured by shape and size for transseptal delivery to the left atrial appendage of the heart via a septal region between a right atrium and a left atrium of the heart.

In Example 16, the subject matter of one or any combination of Example 13-14 can optionally include the control circuit wirelessly coupled to the first stimulation circuit. A wirelessly-controlled electrode assembly can optionally be formed to include the first electrode assembly and the first stimulation circuit as a portion of a wirelessly-controlled electrode assembly configured to be located entirely within the heart. The control circuit can optionally be located remotely from the wirelessly-controlled electrode assembly.

In Example 17, the subject matter of one or any combination of Examples 13-16 can optionally include forming a portion of the first electrode assembly to include a fixation device configured to securely anchor at least a portion of the first electrode assembly within the left atrial appendage of the heart.

In Example 18, the subject matter of one or any combination of Examples 13-17 can optionally include forming a second electrode assembly configured to deliver an electrical stimulation to a non-coronary baroreceptor.

In Example 19, the subject matter of Example 18 can optionally include conductively coupling the second electrode assembly to a second stimulation circuit, and forming a control circuit to control generation of an electrical stimulation by the second stimulation circuit for delivery via the second electrode assembly in coordination with initiating or inhibiting generation of the electrical stimulation by the first stimulation circuit.

In Example 20, the subject matter of one or any combination of Examples 13-19 can optionally include forming a first stimulation circuit configured to generate electrical stimulation therapy specified to assist a blood pressure reduction at least in part via an inhibition of the sympathetic nervous system induced via the stimulation.

Example 21 can include, or can optionally be combined with any portion or combination of any portions of any one or more of Examples 1-20 to include, subject matter that can include means for performing any one or more of the functions of Examples 1-20, or a machine-readable medium including instructions that, when performed by a machine, cause the machine to perform any one or more of the functions of Examples 1-20.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” In the event of inconsistent usages between this document and any documents incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
a first stimulation circuit, configured to be electrically coupled to a first electrode assembly, the first electrode assembly configured to deliver an electrical sub-myocardial activation stimulation to a coronary baroreceptor from a location within a left atrial appendage of a heart, the first stimulation circuit configured to generate the electrical sub-myocardial activation stimulation for delivery to the coronary baroreceptor via the first electrode assembly; and
a control circuit configured to control delivery of the electrical sub-myocardial activation stimulation.

2. The apparatus of claim 1, wherein the control circuit is configured to adjust the electrical stimulation at least in part in response to sensed information indicative of a blood pressure.

3. The apparatus of claim 2, comprising an implantable blood pressure sensor configured to provide to the control circuit information indicative of blood pressure.

4. The apparatus of claim 1, wherein the control circuit and the first stimulation circuit are included in a subcutaneously-implantable housing; and
wherein the control circuit is conductively coupled to the first stimulation circuit.

5. The apparatus of claim 4, further including the first electrode assembly, and wherein the first electrode assembly comprises an intravascularly-deliverable lead assembly electrically coupled to the first stimulation circuit.

6. The apparatus of claim 5, wherein a distal portion of the intravascularly-deliverable lead assembly is sized and shaped for transseptal delivery to the left atrial appendage of the heart via a septal region between a right atrium and a left atrium of the heart.

7. The apparatus of claim 1, further including the first electrode assembly;
wherein the first electrode assembly and the first stimulation circuit are included as a portion of a wirelessly-controlled electrode assembly configured to be located entirely within the heart; and
wherein the control circuit is located remotely from the wirelessly-controlled electrode assembly and is wirelessly coupled to the first stimulation circuit.

8. The apparatus of claim 1, including the first electrode assembly;
wherein the first electrode assembly includes a fixation device configured to securely anchor at least a portion of the first electrode assembly within the left atrial appendage of the heart; and
wherein the fixation device is configured to locate the first electrode assembly in a specified position to deliver the electrical stimulation to the coronary baroreceptor from within the left atrial appendage of the heart.

9. The apparatus of claim 1, comprising a second electrode assembly configured to deliver electrical stimulation to a non-coronary baroreceptor.

10. The apparatus of claim 9, wherein the second electrode assembly is conductively coupled to a second stimulation circuit; and
wherein the control circuit is configured to control generation of an electrical stimulation by the second stimulation circuit for delivery via the second electrode assembly in coordination with initiation or inhibiting generation of the electrical stimulation by the first stimulation circuit.

11. The apparatus of claim 1, wherein the first stimulation circuit is configured to provide a stimulation therapy specified to assist a blood pressure reduction at least in part via an inhibition of the sympathetic nervous system induced via the electrical sub-myocardial activation stimulation.

12. An apparatus comprising:
a stimulation circuit, configured to be electrically coupled to an electrode assembly, the electrode assembly configured to deliver an electrical sub-myocardial activation stimulation to a coronary baroreceptor from a location within a left atrial appendage of a heart, the stimulation circuit configured to generate the electrical stimulation for delivery to the coronary baroreceptor via the first electrode assembly; and
a control circuit wirelessly or conductively coupled to the stimulation circuit and configured to control delivery of the electrical stimulation;
wherein the stimulation circuit is configured to provide electrical stimulation therapy specified to assist a blood pressure reduction at least in part via an inhibition of the sympathetic nervous system induced via the electrical sub-myocardial activation stimulation.

13. A method, comprising:
forming a first stimulation circuit configured to be electrically coupled to a first electrode assembly configured to deliver an electrical sub-myocardial activation stimulation to a coronary baroreceptor from a location within a left atrial appendage of a heart, the first stimulation circuit configured to generate the electrical stimulation for delivery to the coronary baroreceptor via the first electrode assembly;
forming a control circuit configured to control delivery of the electrical stimulation; and
wirelessly or conductively coupling a control circuit to the first stimulation circuit.

14. The method of claim 13, comprising:
receiving information indicative of a blood pressure; and
adjusting an electrical stimulation therapy, using the control circuit, at least in part in response to the received information indicative of a blood pressure.

15. The method of claim 13, wherein the control circuit is conductively coupled to the first stimulation circuit;
wherein the control circuit and the first stimulation circuit are included in a subcutaneously-implantable housing; and
wherein the method further comprises:
electrically coupling the first electrode assembly, including an intravascularly-deliverable lead assembly, to the first stimulation circuit; and
configuring the intravascularly-deliverable lead assembly for transseptal delivery to the left atrial appendage of the heart via a septal region between a right atrium and a left atrium of the heart.

16. The method of claim 13, wherein the control circuit is wirelessly coupled to the first stimulation circuit;
wherein the method comprises:
forming a wirelessly-controlled electrode assembly including the first electrode assembly and the first stimulation circuit as a portion of a wirelessly-controlled electrode assembly configured to be located entirely within the heart; and
locating the control circuit remotely from the wirelessly-controlled electrode assembly.

17. The method of claim 13, comprising forming a portion of the first electrode assembly to include a fixation device configured to securely anchor at least a portion of the first electrode assembly within the left atrial appendage of the heart.

18. The method of claim 13, comprising forming a second electrode assembly configured to deliver an electrical stimulation to a non-coronary baroreceptor.

19. The method of claim 18, comprising conductively coupling the second electrode assembly to a second stimulation circuit; and
controlling generation of an electrical stimulation by the second stimulation circuit for delivery via the second electrode assembly in coordination with initiating or inhibiting generation of the electrical stimulation by the first stimulation circuit.

20. The method of claim 13, wherein forming the first stimulation circuit includes forming a first stimulation circuit configured to generate electrical stimulation therapy specified to assist a blood pressure reduction at least in part via an inhibition of the sympathetic nervous system induced via the stimulation.
